(19) United States
(12) Patent Application Publication
(10) Pub. No.: US 2004/0202615 A1
(43) Pub. Date: Oct. 14, 2004

(54) Melt-processed powder containing particles' repulsion agent, evaporation retardant, and means for benefit to plant cultivation and/or animal husbandry

(76) Inventor: Robert N. O'Brien, Victoria (CA)

Correspondence Address: Robert N. O'Brien, 2614 Queenswood Dr., Victoria, BC, V8N 1X5, CA

(21) Appl. No.: 10/368,747

(22) Filed: Feb. 20, 2003

(51) Int. Cl. A61L 9/04; A61K 9/14

(52) U.S. Cl. 424/46

(57) ABSTRACT

An evaporation retardant composition engineered for wide applicability to co-delivery of non-filmforming chemicals, along with filmformers, wherein the former are as beneficial to plant cultivation and/or animal husbandry in their own uses, as are fatty alcohol filmformers by saving water. A previously disclosed concept of water-induced ionization that causes mutual particles' repulsion to enhance spreading is freshly embodied with use of a dicarboxylic acid, preferably oxalic acid, as the ionic repulsion agent. Avoidance of esterification between the included alcohol and acid infiltration into soil, which is a problem known to limit usefulness of emulsified fllmforming compositions is disclosed.

### CROSS REFERENCES TO PROVISIONAL APPLICATIONS

Two provisional patent applications filed less than a year ago entitle relevant subject matter that is addressed in them, and hereinafter is addressed substantially the same, to the earlier filing date(s) of one or the other or both of the provisional patent applications, entitled: “The Use of a Film Former in Powder Form Spreading on Water to Deliver Desired Chemical Entities”, and “Use of a Film Former in Powder Form Applied to Earth or Crops to Deliver Special Materials”, Robert N. O'Brien, inventor.

### FEDERALLY SPONSORED RESEARCH OR DEVELOPMENT

Not applicable

### MICROFICHE APPENDIX

Not applicable

### BACKGROUND OF THE INVENTION

1. Field of the Invention

This invention relates generally to water conservation using evaporation retardant chemicals, and to manufacturing without significant chemical reactions between constituents of a special powder made by melt-processing blended film-forming and non-film-forming chemicals as hereinafter described, more particularly relating to an end-product that transports the latter along with the former, where the to-be-transported non-film-former is a chemical useful to plant cultivation animal husbandry, or both. Even more particularly, the powder shall contain an agent effective to cause particles of the powder to repel one another giving very rapid spreading. The essential film-forming constituent of such powders is one or more of the straight-chain fatty alcohols well known for use to retard water evaporation.

The powder of the invention is contemplated for usefulness when distributed onto plots of farmland, lake water, gardens, rice paddies, turf of golf courses, or the like, for any purpose of plant cultivation or animal husbandry or both, including fertilization, control of pestilent life forms either plant or animal, desired animal or plant health or growth promotion, faster than usual germination of seeds, and so forth for unusually rapid spreading and delivery of the desired materials.

2. Description of Related Art

U.S. Pat. No. 6,303,133 B1 for “FILM-SPREADING POWDER FOR SUPPRESSING WATER EVAPORATION”, which issued Oct. 16, 2001 to me, Robert N. O'Brien, is the only closely pertinent art that was published containing the ioization enhanced rapid spreading feature before filing this disclosure. The problems resolved by the present invention—and new opportunity seized—would not be faced by someone not endeavoring to make variants of the powder of U.S. Pat. No. 6,303,133 B1 without ruining its unique ionization-enhanced dispersal feature. An official statement of reasons for allowance in part reads: “Applicants have shown that the composition, upon water induced ionization forms a film and spreads on the water surface due to the particles repelling each other. This phenomenon was not obvious from the prior art of record.” That phenomenon, in my opinion, would have to be procured by a powder by anyone else to qualify theirs as closely similar to the powder of my present invention, which I consider an extension of the abovecited and next cited prior inventions by myself.

U.S. patent application Ser. No. 09/739,895 for “COMPOSITION FOR REDUCING EVAPORATION AT SITES BOTH ON LAND AND OPEN WATER”, filed Dec. 20, 2000 also by me received Notice of Allowance on Jun. 4, 2002 but has not issued before filing this disclosure. The chief difference between U.S. Pat. No. 6,303,133 B1 and Ser. No. 09/739,895 is that the former utilizes calcium hydroxide, and the latter calcium sulfate acidified by sulfuric acid, in both cases to afford the “water induced ionization” taught in both disclosures and mentioned in reasons for allowance respecting the first of them to issue as a patent. Neither lime nor gypsum has proved indispensable to procuring water induced ionization, as will be seen.

The total set of new information of my own origination that has been—or if not, soon may be conveyed to the public via the above-identified patent and allowed application is requested to be herein incorporated by reference because nothing would serve better to aid in conveying the present invention than their inclusion in relation to this disclosure. In both the cited patent and patent application the lime and acidified gypsum are required to pass a 100 mesh screen. A guiding calculation assuming cubic powder particles of the bulking agents and taking into account the densities of the two components and quantities of the two present, it was concluded that the averaged depth of the (presumed) encapsulating film former resulting from addint th bulking agent to the melted film former was about 1000 molecules or less thick.

With particular reference now to the cited allowed application disclosing use of “acidified gypsum” instead of calcium hydroxide to afford water induced ionization, I consider the portions of discussion in that disclosure addressed to the desirability of avoiding any significant extent of emulsification to be especially pertinent to one issue that arises in connection with the present invention because of substances proposed to be combined in a melt together, which are known in some circumstances to form an ester that has been disclosed by another inventor to have a function of promoting emulsification.

U.S. Pat. No. 5,518,517 for “WATER-IN-OIL EMULSION FERTILIZER COMPOSITIONS” which issued May 21, 1996 to Jahnke et al discloses compositions intended for distribution onto farmland, gardens and so forth, containing aliphatic alcohols, carboxylic acids, and chemical fertilizer. Because Jahnke et al did not make a powder possessing the ionization-enhanced dispersal feature of my recent U.S. Pat. No. 6,303,133 B1 powder, they did not need to avoid making esters which all competent chemists know can be made by reaction between an alcohol and an acid. They deliberately made such, doing so in order to procure promotion of emulsification, which as already noted is something my compositions are intended to avoid, for a reason explained in Ser. No. 09/739,895 recently allowed, namely: desirability of avoiding impaired water infiltration into soil, which authorities on use of filmforming evaporation suppressants on soils have warned against, and have noticed to be associated with use of emulsions.

Therefore, notwithstanding certain of the same categories of substances are used by Jahnke et al as by me in connection with my present invention, the U.S. Pat. No. 5,518,517 product by them, in which carboxylic acids and aliphatic alcohols are co-reactants in an ester-forming reaction, cannot physically preserve, in their end-product, the specific utility (albeit negatively stated) of avoiding water infiltration impairment as much as one can by avoiding emulsification as much as one can, by avoiding esterification as much as one can—-which in the present case is quite effectively avoided as will be seen. In my opinion, it is not so much an important point to be made that it is onerously difficult to avoid the esterification reaction—the really important point is that the desirability of not making an ester in the first place is not necessarily coming to mind amongst those who like Jahnke et al are working with similar substances as I do, developing compositions for broadly similar purposes too. Processing conditions significantly account for differences in result, in view that comparatively long periods of being held at a high temperature are practiced by those who, unlike me, intend to make esters out of carboxylic acid and aliphatic alcohol co-reactants.

Long familiar with esterifications reacting acids and alcohols R. N. Swindlehurst and I once published a Note entitled “Molecular weights by infrared spectroscopy”, Canadian Journal of Chemistry, Vol. 45, 2856-2857, 1967, describing our making an ester of “an unknown alcohol” in order “to find the molecular weight by comparing its absorbance with that of known esters . . . ” Today, thirty-five years later, I find myself having to carefully avoid blending aliphatic alcohols and dicarboxylic acids in a manner making esters, not only because of the afore mentioned emulsification-related water infiltration impairment problem, but fundamentally because an unmodified character of the straight-chain fatty alcohol is worth preserving, for reason of better evaporation reduction.

The common soil conditioners of agricultural lime and gypsum have already been shown in my previous works cited above (and requested to be incorporated by reference) to be blendable with aliphatic alcohols in a melt with the wholly satisfactory result of facilitating manufacture of two powders each of which procures the water ionization enhanced dispersal effect in the end-use. Regarding other soil treatment chemicals, including five fertilizers to be identified shortly, however, even my own published works do not specify how to incorporate them without any unwanted chemical reactions. Similarly, for other candidate cultivation/husbandry chemicals I intend to spread at the same time as a fatty alcohol film that retards evaporation, there have been technical risks involved, especially in connection with the sulfuric acid present in the “acidified gypsum” of the powder disclosed in the abovecited recently allowed but not yet issued patent application entitled “COMPOSITION FOR REDUCING EVAPORATION AT SITES BOTH ON LAND AND OPEN WATER”.

I recently re-evaluated the constituent called “acidified gypsum” and concluded that it would be unreasonable to expect always avoiding degradation of the dispersal-enhancing end-use feature procured via the contained sulfuric acid, if a wide range of additives were to be contemplated for presence in a melt with it. It works with gypsum, but even then at risk of esterifying the fatty alcohol, and it is not of sufficiently wide applicability compared to what I have wanted to do, and now can, after discovering a better agent to procure the particles' repulsion effect the ionized sulfuric acid desorbed from pre-acidified gypsum produced.

The old “acidified gypsum” incurs loss of at least some of the sulfuric acid in it, if potash (potassium chloride) is blended into a melt of aliphatic alcohol containing the gypsum acidified by pre-mixed sulfuric acid. Not easily noticed, especially when melt temperature may not have been above about 100° C., a chemical reaction between some of the sulfuric acid and some of the potassium chloride occurs, producing just a small quantity of hydrogen chloride gas that has not been a major problem in the very small-scale batch-type experimental production runs I have conducted in open air conditions, i.e., without any tailgas recovery means to absolutely prevent release of any hydrogen chloride whatsoever to the environs. However, this occurrence would become a significant problem for large-scale production, necessitating unwanted costs respecting tailgas recovery that will not be a factor for the present invention.

Furthermore, the general reactivity of sulfuric acid with other substances is too ubiquitous to free up for maximum advantage use of the water ionizable feature of the former inventions to transport a wide variety of plant cultivation and animal husbandry chemicals at the same time as spreading evaporation retardant.

### BRIEF SUMMARY OF THE INVENTION

Regarding an “Object of the Invention”, this is primarily to extend the utility of evaporation retardant-containing powder compositions capable of enhanced dispersed by means of the ionization-procured particles-repulsion feature of the prior art of my own origination as heretofore cited and requested to be incorporated by reference. I propose to extend the utility of such compositions by incorporating into a melt-processed powder-making blend of filmforming and non-filmforming chemicals, at the melt-processing stage, further special chemical entities that are normally useful as agricultural or aquacultural materials when they are distributed by themselves onto surfaces of agricultural or aquacultural sites, meant as including farmland, lakes, gardens, ponds, lawns, aquaria, golf courses, rice paddies, and the like. Examples of such special chemical entities are the fertilizers: potash, ammonium phosphate, ammonium sulfate, ammonium nitrate, and calcium superphosphate.

In order to secure the utilitarian object of the present invention it is important to suppress likelihood of any reactions among substances in the melt that could so change the substances in it (potential co-reactants) that the utilities associated with each of them in the end-use of the powder made by melt-processing would be degraded. For example, chemically changing the straight-chain, non-sidebranched feature of an aliphatic alcohol such as hexadecanol or octadecanol is known in the art to degrade its evaporation retardancy, and this was known even before my recent inventions in the field of evaporation retardant compositions. Furthermore, and here becoming significant only because of the nature of my above-referenced recent inventions, especially with respect to my application Ser. No. 09/739,895 continuing with subject matter of U.S. Pat. No. 6,303,133 B1 in part, when acids and alcohols are mixed in a highly heated circumstance it is only reasonable to expect a prospect of some reaction which would of course involve changes to both the acid and the alcohol involved.

The primary object of the present invention has also had to be met in the face of the complication and technical risk incurred by incorporating substances like the abovenamed five fertilizers into a melt containing an acid for the end-use dispersal feature and an aliphatic alcohol for evaporation retardancy, since the acid could conceivably react with the incorporated substance. Not only mentioned above herein, but in fact of Record, I raised the point in the second to last paragraph of my written description for application Ser. No. 09/739,895: that blending potassium chloride (potash) into a melt in which sulfuric acid is present would involve an “expected tendency of producing gaseous hydrogen chloride”. The present invention gets around that as well as the other problems.

Accordingly, through a course of non-routine experimental tests of various examples of film-spreading powders in which I substituted a dicarboxylic acid for the original sulfuric, with continued use of recognized fatty alcohol for the film-former, and using some ground gypsum as a particles building constituent, I incorporated into each of five powder-making melts—a different one each time—one of the five above-named fertilizers with good success. The presently disclosed invention meeting the above stated object of the invention, and other desirable objects, has thus been made, the nature of which may be summarized as next follows.

The homologous group of dicarboxylic acids all having melting points in a range above 97° C. is hereby specified as particularly suitable from which to select an acid for the purpose of blending into an above-room-temperature melt of normally solid phase higher aliphatic alcohol having a long, straight, unbranched carbon-containing chain when the melt also contains non-filmforming constituents that could include ground gypsum, but need not and may now include one or more than one previously unmentioned agricultural or aquacultural chemicals useful to distribute with the evaporation regardant, eg., onto farmland, lakes, gardens, rice paddies, golf courses, etc., provided conditions of blending in the melt are so arranged and melt-processing is so conducted that no significant chemical reactions occur to degrade constituent properties.

The role of dicarboxylic acid in the end-product film-spreading powder composition containing an agricultural or aquacultural chemical as well as an aliphatic alcohol evaporation retardant is the same as that of lime in abovecited Pat. No. 6,303,133 B1 and of sulfuric acid absorbed in the gypsum of application Ser. No. 09/739,895. When the end-product contacts or is contacted by water, the acid will undergo an ionization that leads to temporarily held like charges on the composite particles of broadcastable particulate material of which the end-product consists, the particles thereby tending to mutually repel one another. It has become apparent from tests that oxalic acid has the best dissociation constant, is convenient and hence is the “preferred” dicarboxylic acid I recommend using. Under the conditions of melt-processing to be described below, the co-presence of oxalic acid and aliphatic alcohol is, as desired, free of significant risk of an esterification reaction occurring. Furthermore, with none of the five fertilizers tested has there been any indication of reactions between the carboxylic acid and any of them, in contrast to there having previously been (some indication of) an undesired reaction between sulfuric acid and potassium chloride. Pesticidal additives addressed below are highly likely so to be incorporatable, hence I am satisfied that the present invention meets its object of extending the range of substances to be co-spread with alcohol films.

### DETAILED DESCRIPTION OF THE INVENTION

When water is contacted, irrespectively of whether in an accumulated reservoir thereof or in soil pores and coating soil particles, oxalic acid disperses fatty alcohol film spreading powders faster than either slaked lime or acidified gypsum.

From the chemical structure of calcium hydroxide, its hydroxyl ions probably come off in sequence, inhibiting one another somewhat from doing so apparently. With oxalic acid and other acids of its homologous series (dicarboxylic acids), there is better distancing between the hydrogens compared to the hydroxyls in lime, hence essentially no mutual inhibition to peeling off during ionization, thus giving two positive charges at a time per molecule of oxalic acid in contact with water from the edges of a composite particle containing transported entities, fatty alcohol, and molecules of oxalic acid to generate the mutual repulsion effect originally discovered and disclosed in connection with slaked lime in admixture with a fatty alcohol.

The result here is greater repulsion and faster spreading as long as the composite particles with like charges so acquired exist. Of course, as the transported entities drop out and the filmforming alcohol ceases to be particulate itself by forming a monomolecular scale two-dimensional structure, the earlier charge carrying phenomenon will have done its job before ceasing to be a factor.

Even though I originally thought of trying oxalic acid as a substitute for sulfuric acid in the pre-mixed “acidified gypsum” constituent used in the allowed application's special composition, I also actually tested, in rudimentary manner, the effectiveness of oxalic acid respecting enhanced composition dispersal even when the original lime is present, not gypsum. I made a composition containing lime, technical higher fatty alcohol combining hexadecanol and octadecanol, and some oxalic acid, the lab-made composite particles being large enough to see at two meters distance. Dusted onto the surface of my large water-filled pool, the particles in three to five seconds were moving apart visibly. It was reasonable to expect the known lime ionization factor to work over a long timeframe, so I was not surprised to observe spreading an hour and a half after dusting. The surprise was that a half hour after dusting still visible particles were coming together for a brief period. I attribute very fast spreading at the start of the experiment to the effect of the oxalic acid, the much later motions to lime. At a point in time between, I believe there occurred a canceling-out positive versus negative charges effect temporarily robbing the powder particles of mutual repulsion because both lime and oxalic acid were ionizing. Mixing lime and oxalic acid is not a better alternative to straight lime as in the original patented powder, but oxalic acid without lime I do propose.

Advantages of Oxalic Acid Over Using Sulfuric Acid

The advantages of oxalic acid over using sulfuric acid, moreover, are of major significance. The latter is very hygroscopic so that unless a composition containing it is carefully protected from contact with moist air there will be water entering the composition. Then, in the presence of water, sulfuric acid forms a half-ester with the fatty alcohol—a half-ester because only the first hydrogen is really active, but there may be a small quantity of a double ester formed as well. As indicated above it is not desirable to form esters in a composition intended to retain optimum evaporation retardancy, and the other reason is that esters present give the composition a tendency to emulsify, also not desired. Furthermore, the off-gassing reactions with potential chemical fertilizers, specifically potash, are a problem with sulfuric acid, but not with oxalic acid or other members of its homologous series. It is the best among the dicarboxylic acid series, for the present employment, because it has the highest dissociation constant of the lot. The lower dissociation constants accompanying higher molecular weights means ability to ionize hydrogen ions and procure the desired effect therefrom goes down.

Its moderately strong acidity couples with modest solubility in an advantageous way, too. Oxalic acid is a solid, doesn't react with solid alcohols or gypsum, and remains as the anion to charged particles to the end or nearly the end of their meaningful existence as composite, like charged, mutually repelling structures. The rapid dissolution of sulfuric acid can cause its disappearance before that of the composite particles which are intendedly spread whereas the particles' repulsion effect from oxalic acid persists longer, if not so long as lime. Further, the sulfuric acid, although supplying a hydrogen ion for dispersion of the like charge particle really only ionizes one hydrogen ion, the second being about 100 times less ionizable, where oxalic acid as noted probably ionizes both simultaneously giving a doubly negatively charged particle and hence more speedy spreading of the monolayer. Organic, biodegradable oxalic acid is not nearly as hazardous to work with as is sulfuric acid, and this with all the foregoing reasons makes it a remarkably suitable powder constituent to provide the same kind of already previously disclosed water induced ionization powder particles to repel one another. Of critical significance is the fact of discovering that a dicarboxylic acid, preferably oxalic acid, can be used as the active repulsion-causing ingredient together in an evaporation suppressing composition based on higher fatty alcohols with a wide range of included to-be-transported non-filmformers from classes of chemicals having known utility in cultivation of plant life and animal husbandry, both marine and terrestrial for both categories. Oxalic acid is a good chelating agent and will chelate most doubly charged ions such as calcium. However none of the fertilizers mentioned above have doubly charged ions except calcum superphosphate. The solubility of this fertilizer is in the same range as that of gypsum and so it is to be expected that some of the superphospate will be associated in solution with a chelated calcium ion. However this does not hinder the plants ability to utilize the phosphorous supplied by the superphosphate I have reiewed lists of pesticides, algicides and herbicides and find none that have doubly charged ionizable cations and so anticipate no difficulty in distributing such materials on water and soil by the present invention Before illustrating the kinds of to-be-transported constituents, the matter of a major change concerning inclusion of pre-sized solid phase particles in the melt for making the composition needs to be addressed.

The original two evaporation suppressing compositions, using powdered lime in one and powdered gypsum in the other, took multi-functional advantage of the pre-particulate nature of these constituents. On one hand, ions-releasing dissolution of each, though extremely modest for gypsum (reason it needed sulfuric acid added), contributed functionality in the water-induced ionization mechanism responsible for mutual composite particles' repulsion. On the other hand, the same pre-particulated constituents being non-melting in processing, contributed to formation of composite powder out of the fatty alcohol melt. Oxalic acid, the replacement ionic repulsion agent, does not itself melt at the processing temperatures of fatty alcohol melts for making the present end-product; however only a small amount is needed to procure the particles' repulsion effect. I do not suggest oxalic acid in the earlier taught large proportions of lime or gypsum.

Since neither powdered lime nor powdered gypsum powder is any longer required as a starting material, the opportunity arises to select one or more replacement substance(s) directed specifically to two roles: (one) facilitating powder formation from the melt, and (two) assisting to maintain the composition for an appropriate period of time on any water surface contacted. A reliance on surface tension to buoy up denser-than-water composite particles of mixed composition has been resorted to previously, and remains feasible, but greater assurance that particles will not prematurely sink (if at all) before being scattered amidst spreading film can be obtained by choosing, when appropriate, an agent for role (one) just above that also serves role (two).

I appreciate that others have proposed crushed vermiculite, fly ash, or various low weight units as bulking agents in evaporation suppression compositions. There is no reason here to not include the same, although in a new combination. Hollow glass microspheres may now be advantageously blended with molten fatty alcohol and solid-phase oxalic acid to make the unique end-product composition of the present invention. By judicious procurement of bulking agent starting materials that are lightweight, even more advantage is derived from the ionic mutual repulsion effect than when higher density lime and gypsum particles were used. Pushing a visible composite particle up the slope of a ripple in a water surface, or surface of hemispherical water drop on a plant's leaf, looked at closely, is expected to be easier for a lightweight-cored particle.

Accordingly, since the repulsion-causing ingredient is now not needed to be present in large proportion for a given amount of manufactured composition, a fine particulate bulking agent that stands up to the melt conditions like gypsum but not necessarily as dense may be used as a composite particle's building component, units of which are cores or carriers to which are temporarily adheded filmforming and ionic repulsion inducing components.

The bulking agent, which still could be ground gypsum although not with absorbed sulfuric acid, can be hollow glass microspheres, hollow atapulgite beads or a dozen other things, non-melting at the fatty alcohol melt temperature, not readily dissolvable in water, scaled to the particle size wanted, non-toxic if ingested by livestock, chemically unreactive with either the filmformer or transported cultivation/husbandry chemical, and preferably inexpensive. There exists a substantial repertoire of such materials today, with no problem of acquiring them.

The above concept of doing without lime or gypsum as multifunctional bulking agents, by substituting in their places something else to procure the particles-building function for the product derived from the melt, encompasses non-preferred embodiments thus far. They are workable and allow a four-component composition that is quite useful to be made, where the four components are: 1. the fatty alcohol filmforming constituent; 2. the dicarboxylic acid particles repulsion agent; 3. a particles' building pre-particulated bulking agent—which may be inert microspheres; and, 4. one or more than one cultivation/husbandry chemical.

However, the ultimate and preferred embodiment of the present invention resides in a three-component melt-process made composition, wherein the three components are: 1. the fatty alcohol filmforming constituent; 2. the dicarboxylic acid particles' repulsion agent; and, 3. one or more than one chemical for cultivaton/husbandry, prepared in a suitable fine particulate form so that it (or they) can function as the pre-particulated bulking agent non-melting in the melt, as well as being the to-be-transported material spread simultaneously with a fatty alcohol film.

It is known that many chemicals for plant cultivation and animal husbandry uses are available in fine particulate forms. Some either are or could readily be made as lightweight spheres or beads, solid or hollow, so I propose taking such a particulated chemical that is (1) able to withstand being in a melt of fatty alcohol without damaging either its (a.) physical form or (b.) end-use function for cultivation/husbandry, and is (2) chemically non-reactive in the melt with anything, and then making instead of a four-component non-preferred embodiment—the preferred three-component embodiment at my suggestion, which will attain with negligible difficulty obviation of needing use of fly ash, inert microspheres, ground vermiculite, powdered gypsum, or any such material needed in four-component versions of the invention solely to furnish the particles' building agent—ie. “bulking agent”—in the melt.

Regarding a fourth component in cases where the cultivation/husbandry chemical desired to be delivered in accordance with the method of the invention is a cultivation/husbandry chemical with same tendency to violate one of the principles “(1a.)”, “(1b.)”, or “(2)” just enunciated above, a fourth component should be selected with a view not solely to serve as a particles' building—i.e., “bulking agent”—but additionally, as mentioned somewhere in one of my provisional applications, selected for ability to render the to-be-delivered chemical entity substantially compatible (whereas it otherwise would not be) with the proposed melt-processing to be enacted in carrying out the invention, restricted to substantial compliance with the enunciated clear principles. An example is next supplied by reference to liquid pesticides.

It has long been known as a conventional practice in the plastics industry to incorporate antimicrobials, mildewcides, fungicides, bacteriocides, or other biocides, as preservatives in articles made from polymeric resins. The practice naturally resulted in the identification of numerous chemicals that withstand the typically rather severe conditions of polymeric melts. Many if not most such plastics preservatives are liquids when added to resin blends for processing into solid articles. Although their uses do not by design include their release to the environment, the prospect of that happening is real enough that they must be considered pesticides, to be registered with the EPA (Environmental Protection Agency). They certainly will be also, when by design incorporated into powders broadcast on land and water.

I suggest that liquid pesticides the same or analogous to those used in the plastics or other industries may be incorporated into powders embodying the present invention. Because a favored three-component powder requires a large proportion of unmelted solid phase material in the processing melt of fatty alcohol, such liquids cannot serve as the “bulking agent” which helps build composite powder particles out of the melt, recalling here the intention not to use so much oxalic acid (non-melting itself) that there would be enough of it to serve as the bulking agent. The aforesaid suggestion of inert microspheres or the like to take over the bulking agent role should in this case be combined with the suggestion of incorporating liquid pesticides. A resulting embodiment of the “non-preferred” (because more complex) nevertheless highly valuable “four-component” type results, comprising: 1. the fatty alcohol evaporation retardant; 2. the dicarboxylic acid, preferably oxalic acid, as the particles' repulsion effect agent; 3. an inert solid phase, non-melting-in-the-melt, particulate particles' building agent, eg., microspheres, if not ground gypsum; and, 4. a suitable liquid pesticide the end-use effectiveness of which must not be compromised by undergoing the melt-processing conditions. Element “4.” is readily procured, as plastics preservatives from the same industry most involve in using microspheres, as well as usual pesticide sources. This liquid, or any desired liquid can also be made to a powder separately with the bulking agent and the two powders mixed in the preferred ratio in the well-known powder blending operation shown in Chemical Engineering texts.

One of many candidates that more likely than not would work against microbial pests if released from a powder embodying my invention is “2-o-octyl-4-isothiazolin-3-one”, which at an active ingredient level of only 0.04-1.0% in plastic pool and pit liners significantly slows down their degradation by natural living organisms in water. Since this chemical stands up well to plastics processing conditions, no competent chemist would doubt that it can survive 50°-100° C. in a fatty alcohol melt with oxalic acid and enough microspheres that the quantity of liquid used does not make an undesirable paste instead of the powder that is wanted. A similarly functional anti-microbial pesticide is “10, 10′-oxybispheno-xarsine”, used in shower curtains and plastic roof membranes. Microbial degradation is problematic not only underwater but almost everywhere. The foregoing and other plastics-industry pesticides are old art I saw listed in a ten year-old Mid-October Issue of MODERN PLASTICS ENCYCLOPEDIA '92. They are mentioned merely as examples that neither confine the invention nor have to be used, but prove liquid pesticides could survive in the melt or be incorporated as separately prepared powders for ultimate mixing.

The point is that routineers in the art can be confident they would find it easily within their existing level of competence to search out suitable liquid pesticide candidates for inclusion in a powder embodying my invention in its four-component version, wherein an inert solid-phase particles' building agent (or ie., “bulking agent”) is present in order—additionally to promoting powder formation from the melt—to absorb a cultivation/husbandry chemical that is not necessarily solid phase itself. It may be liquid. The cultivation aspect is evident in any use of pesticides or herbicides intended to control organisms one has no intention of raising, since after all they obviously compete in use of a given environment with whatever it is that one does intend to raise. What I have been calling “cultivation/husbandry chemicals”, in other words, has for the purposes of my disclosure the non-repugnant sense of including pesticides and herbicides, as well as fertilizers, growth hormones, livestock pharmaceuticals and all other candidate materials to be first blended in a melt and for later “delivery” along with evaporation retardant to end-use sites, as initially disclosed in the two provisional patent applications. An exception “candidate” is the bacillus thuringiensis israeliensis and close relatives that are effective for controlling mosquitoes. These living pesticides have since been made the included topic of a different invention. Since roughing out the provisional application mentioning it, I read something from which I inferred that the spores would be at risk of being lysed (de-shelled, so to speak) and injured, if cooked alive in one of my melts as I had originally contemplated, before deciding that living things are better left uncooked. It therefore further clarifies the scope of my definition of “cultivation/husbandry chemicals”, to exclude living biological agents of pest control, unless they can be prepared as a separate powder for later mixing in the proportions needed. It here should be emphasized that in some cases the second powder approach is desirable even if the material to be spread is solid and can endure the melt process since for pest control, whether flora or fauna may require varying doses of the transported material as the season progresses.

Certain prophetic examples of applying the invention to aquaculture and agriculture, plus one pseudo-agrarian activity, will next be presented.

Illustrative Operation of End-Product to Fertilize Submerged Planting

Many useful seaweed species may be cultivated in gravel or soil-like growth media underlying a body of water maintained in a pool substantially free of extensive under water currents, and such plantings are desirably fertilized by an evenly distributed scattering of suitable chemical particles, without necessity of a hypothesized “lead-booted underwater fanner” walking along the bottom, if not riding some kind of submarine-tractor. The instant invention can be used and it will at the same time provide an evaporation suppressing film at the water surface, so that replenishment of the liquid volume of the pool to replace evaporative water loss may be conducted at more widely spaced than usual intervals of time.

All that is needed for such an operation as fertilization of submerged planting is a preferred version of the end-product of the powder-forming process of the invention that contains: (1) a recognized fatty alcohol evaporation retardant; (2) oxalic acid to provide the mutual particles' repulsion effect; and (3) fertilizer particles that are sufficiently dense when stripped of temporarily adheded fatty alcohol and oxalic acid that they will not remain uselessly suspended by surface tension atop the pool. Component (3) as extensively explained above is here assumed capable of having survived the melt-processing conditions without harm to itself or anything else, or having been prepared as a separate powder for later mixing.

Those who contemplated denser-than-water particles supported at a water surface by surface tension, in association with simultaneous formation of a fatty alcohol film on the same surface, sometimes inadvertently forget the well known fact that the spreading of such a film entails reducing the surface tension property of the surface. Not to say I am the only worker to have taken advantage of the fact for some kind of useful purpose, I have, in connection with a separate invention for detecting clandestine biological warfare attacks, suggested monitoring among other things the change in surface tension brought about by loading the surface with toxic anthrax powder, however, changing surface tension is resorted to differently. The plan of the operation here is to ensure an ultimately even distribution onto the submerged planting, of comparatively heavy seaweed fertilizer particles that are first to be evenly distributed in the plane of the reservoir surface, whence they subsequently fall or disolve.

Were it not for the fatty alcohol and oxalic acid working in harmony to produce an intended lateral motion effect, such fertilizer particles would sink straight down immediately after being cast upon a portion of the reservoir surface, in the event surface tension were inadequate to support them. Because of the effectiveness of the instant invention, however, a powder containing such particles with fatty alcohol and oxalic acid temporarily adheded thereto can be cast upon a limited area of the water surface, and its composite powder particle constituents will become scattered apart from one another very rapidly because of the charge they are carrying, stretching out to cover the larger area. The specially made composite particles do not sink immediately, but any one will sink when stripped of the temporarily adheded fatty alcohol and oxalic acid carried by it. The function of such stripping is performed by water, but not in so fast-acting a manner as the function of lateral motion induction due to ionic repulsion-enhanced rapid spreading. The rate of spreading is also enhanced by the lack of crystal lattice energy in the film former. A calculation for the lime based product, where the lime particles must pass a 100 mesh screen, shows that at a ratio of 10/1 lime to film former, the average thickness of the film former that the melt process produces is approximately 1500 molecules. Since as the size of a particle decreases, the area of say a cube or sphere decreases faster than the volume, the thickness of encapsulating film former in fact, slowly increases Such an encapsulating layer can only be described as either amorphous or glassy, which in either case is a higher energy state than the crystalline state and therefore should allow faster spreading of the film former to a monolayer on the water than from a crystal. A trial on my pool with oxalic acidified gypsum gave a spreading speed more than ten times faster than particulate 1-hexadecanol.

It is not just the loss of the contribution to buoyancy owing to stripping off fatty alcohol detachment that is operative; the film being formed reduces the surface tension of the water surface spread across, so that even if a denser-than-water small particle might other wise be supportable by normally occurring surface tension, the artificially induced reduction of surface tension in areas where the fatty alcohol film becomes established allows the sinking of so-stripped particles, at the same time not-yet-stripped counterparts (still combined in form of composite particles) continue in the scattering motion for a while, until ultimately all particle scattering upon the film-modified water surface must cease because everything else has dropped out and only the spread film no longer particulate in character—is left on the surface. It should also be remembered that to be available for plant use, a fertilizer must have minimum solubility. All of the inorganic fertilizer are at least slightly soluble and all have an affinity for water or will drop through the surface when the encapsulating film former is removed during spreading.

My previous disclosure in FILM-SPREADING POWDER FOR SUPPRESSING WATER EVAPORATION, U.S. Pat. No. 6,303,133 B1 adequately described and explained how the fatty alcohol film spreads between water-alterable composite particles that start out as combined solid phase fatty alcohol and calcium hydroxide (slaked lime) but that undergo change as the combination is disassembled, so to speak, by its contact with water. Here the same change to and disassembly of composite particles occurs, but wherein a more persistently particulate component survives I believe, as a stripped-bare fertilizer particle long enough to sink intact, downwardly through the water to the reservoir bottom, where, for the particular illustrative example presented, a submerged planting of a cultivated seaweed can be fertilized by application of the present invention. It is to be expected that some of the fertilizer will dissolve but that most will arrive in the vicinity of the crop.

Applications involving an intention to sink particles for delivering fertilizer to a submerged planting are furthered by recognizing the important role of surface tension reduction accompanying film formation, the same feature of alcohol films used to detach mosquito larvae from water surface suspension.

Illustrative Operation of End-Product to Dose Livestock Drinking Ponds

Livestock raised in certain regions tend to suffer certain mineral deficiencies associated with the mineralogy of the soils of the region, and so it is known to dose drinking ponds for the livestock with needed mineral supplements. Here is an application to which the present invention is easily turned. The mineral supplement need only be provided in a suitable form, inert in the melt process making the composite powder of the invention, and that will not be injured by the 50°-100° C. heat of the melt process, or again it can be prepared as a separate powder with the bulking agent to be added in appropriate amounts to the main water evaporation retardant powder. The mineral, with or without the bulking agent as required, either natural or synthetic and finely particulate, combines in this case with a small amount of oxalic acid and an amount of fatty alcohol sufficient to simultaneously provide an evaporation suppressing cover of film on the drinking pond that is to be dosed with the livestock health-promoting mineral. Routine art using such films calls for using an excess so that film self-repair is viable. It may also be noted that one does not in this case want the delivered husbandry particles to accumulate on the bottom of the pond. They should either float within reach of drinking animals to be ingested with water, or else dissolve so that all water ingested contains at least some of the supplement. However a rough calculation suggests that if the bulking agent is gypsum (a slowly dissolving material) that using the powder as directed, that is twice per week if saving water is also a concern, it will take 500 years to produce a layer one centimeter thick assuming no gypsum dissolves. In cases where microspheres or absorptive plastic beads are to be used, either pushed along by the expanding film the resulting layers of material on the bottom of ponds will increase faster if the conveying beads sink.

Pseudo-Agrarian Operation of End-Product in Plastic Turf-Tending Disinfection

Plastics materials are known which contain anti-microbial agents and clearly such materials can be used to manufacture plastic synthetic turf formed with millions of erect ersatz “grass blades” per hectare. No single agent is effective against every conceivable infective microbe of course, so a circumstance that can occur is an infection by microbes requiring to be dealt with independently. Water containing a suitable disinfectant might be flooded over a germ-infested horizontal ground cover consisting of synthetic turf, but a lot of water would be unnecessarily wasted by such flooding, now that I have made the present invention. Instead of flooding, a light sprinkling or dusting of the specially made powder is first applied, then a light watering to allow the spreading agent to make the anti-microbial agent to spread on the “grass” blades.

The disinfectant, preferably in pre-powdered form, should be blended in a melt of long-chain fatty alcohol together with a minor proportion of oxalic acid, to make a powder for disinfecting artificial turf on which people may play football, if not range their chickens (also viable). Powder applied spreads on the water droplets on the “leaves”. Water actuates oxalic acid to perform its particles' repulsion function. Fatty alcohol spreads its film with all that this entails, such as surface tension reduction. The contained disinfective component delivered for service in this use-intention function, as in the others, will effectively be distributed across a surface of liquid water, though here probably curved.

Shortly after contacting a drop and simultaneously with film-spreading, the fatty alcohol constituent causes its usual surface tension reduction effect, whence the drop will acquire an increased tendency to spread and to fall (by gravity) and its shape will be modified by the change in surface tension plus presence of particles. Consequently, a disinfectant component in the special powder will be more effectively spread down into creases between blades of plastic grass than if the same powdered disinfectant were distributed without fatty alcohol. Worth noting is that natural dewdrops resting well aboveground on natural vegetation can be caused to drop off and go down to soil, by judicious dusting with a fatty alcohol-containing composition, thereby not allowing the dewdrops to merely evaporate in position, but instead, after a fashion, promoting their collection by the soil.

Once those already of general skill in the art grasp the dynamics of powder uses, where all uses depend ultimately on bringing the special composite powder of the invention into contact with water, the invention will probably find numerous applications I have not even begun to contemplate. Like all those already mentioned in the two provisional patent applications, pertaining to delivery of such “candidate” materials as fertilizers, pesticides, herbicides, growth hormones, zebra mussels controls, and so forth, all instances of uses of the present invention exploit the faster spreading, anti-clumping feature brought about for film-spreading alcohols by their combination with chemical agents for water-induced ionization causing a mutual particles' repulsion effect. And hence rapid spreading.

Regarding the evident option exercisable when determining whether to choose either a pre-powdered solid phase candidate material for delivery by means of this invention, or else a liquid candidate made into a powder component by absorbing into or onto material such as vermiculite or microspheres, it will be common-sense and rational analysis of conditions of use that dictate the choice. In the above example illustrating disinfection of artificial turf, a moment's reflection makes it clear that glass microspheres as absorbent material to carry a liquid disinfectant would not be appropriate. After a certain number of disinfections, the microspheres could conceivably accumulate to so great an extent amongst the plastic “grass blades”, that the traction quality of the artificial turf could be adversely modified. Then, when the football players, or whomever, begin to fall down a little more often than usual because of inferior traction, the microspheres may do some injury to the health of the players. These kinds of concerns are routinely addressable in connection with any and every proposal to insert some new composition to human-used environs, and uses of the present invention are no different.

Routine Chemical Screening Procedures

The state of the art in chemical engineering being so high today, I consider it acceptable to leave it to the routineers of that art to go over the chemicals and chemistry principles with which they are familiar, with a view to selecting suitable candidates to be constituents together in new powders embodying the present invention with fatty alcohol film-spreading content enhanced by co-presence of a dicarboxylic acid particles' repulsion agent. An experiment to ascertain inertness in the blend at the melt-processing stage of manufacturing the end-product of this invention involves no more than routine analyses before and after that stage. If any trial component in the melt is not inert, the resulting chemical change will show up in subsequent analysis. Though such experiments are needed, and I have not done them except with a relatively small repertoire of five candidate fertilizers, the experiments are routine in nature and not unduly straining of the level of chemical engineering skill available.

The five fertilizers I know will combine inertly in a melt with oxalic acid, with gypsum as a bulking agent (no sulphuric acid), and commercial grade hexadecanol-octadecanol fatty alcohol material, are ammonium nitrate, ammonium sulfate, ammonium phosphate, calcium superphosphate, and potassium chloride.

For five similar batches staying with about a 10/1 weight ratio of non-filmforming constituents to fatty alcohol content, I substituted oxalic acid for the sulfuric acid formerly used to acidify gypsum, and replaced a significant percentage of the gypsum in each test batch with one of the five fertilizers. Neither the melts nor end-products stank of chemical reaction by-products, as had potash in a gypsum/fatty alcohol blend wherein the gypsum had been acidified formerly using sulfuric acid. When thrown onto my pool surface, all the end-product powders spread very rapidly. It was already known that the calcium superphosphate and ammonium nitrate are useful to fertilize cultivated oysters and algae fed up on by Tilapia fish, thus although I was not at the time cultivating oysters or raising Tilapia in my pool, I knew I would be able to effectively apply fertilizer in aid of those activities, using the new powder invention, if I wanted to.

Because I am now proposing an invention of wider applicability than its precursors out of the same lab, I do not wish to confine it to particular formulations respecting weight percentages of constituents to be used. After all, the candidate materials for delivery by means of fatty alcohol-containing powdered compositions may be wanted in widely varying forms and doses for very diverse applications.

A transported insect bait to kill pests eating it may be required to remain attached to vegetative leaf material above ground until eaten, a totally unlike circumstance to either causing dewdrops to fall to soil, or fertilizer particles to drop to the bottom of a pool. The densities and even the sizes as well as other physical aspects of such candidate particles would be expected by nobody to coincide. It is true that the carrying capacity of the film-forming fatty alcohols, even when (as here) enhanced by inclusion of oxalic acid and a bulking agent (that can be the delivered material itself, or not), is certainly not unlimited. However, again as with a need for some test-screening to rule out unwanted reactivity in the melt, the needed information to arrive at various formulations suited to different end-purposes will—in my judgement—prove to have already been within ordinary chemical engineering skill level to acquire by routine test batch making and experiments that would not have been undue even before publication of this disclosure, let alone after I have herein supplied motivation to make the new batches and do new experiments.

Composition Manufacture

On the remaining topic of indicating the recommended manner of making a composition in accordance with the present invention, considerable reliance may be placed on the pertinent information incorporated in the issued patent and soon-to-be-issued patent application identified above and requested to be incorporated herein by reference.

A basic pair or alternative processing methods warranting description here consists of one method for batch-type powder manufacture and another for large-scale mass production. The batch-making method is easily conducted by melting the fatty alcohol constituent, then stirring into the melt the other constituents. The preferred large-scale method is to have all the starting materials initially in pre-powdered forms, including the fatty alcohol, and if a liquid chemical for cultivating/husbandry purposes in the end-use is desirably included then even that liquid should be pre-absorbed into a powered inert bulking agent constituent, so that all the starting materials are powders at the moment a massive hot air blowing operation is applied to mix them all together simultaneously as the fatty alcohol melts. The hot air melt process is already common and is controllable so that melt temperatures need not be so extreme as above quoted, indeed a temperature of as little as 10 degrees Celcius above the melting point of approximately 55 degrees Celcius may be possible with subsequent saving of energy and heating and cooling times.

I want to forestall a possible concern that blowing heated air through the starting materials may oxidize some of the fatty alcohol, converting some of it to a long-chain fatty acid. I assure the artisans that this is no cause for worry, in view that my laboratory long ago established that even faster film-spreading speeds are achieved if a small amount of long-chain fatty acid is mixed with the fatty alcohol of similar carbon chain length. An experiment with about 5% C14 acid with 1-hexadecanol spread 25% faster than the pure film former.

When conducting large-scale composite powder manufacture using the blown hot air, motionless mixers comparable to those used in blending resins in the plastics industry, and which are also comparable to sieving and attrition bars for sizing dry particulate matter, can be set up in the path of the stream of hot blown air carrying the pre-powdered starting materials. A mid-air adhesion process joining the various constituents by virtue of the molten fatty alcohol will be highly effective, requiring minimal work to get the product finished enough to allow it to fall directly into sacks, drums, boxcars or the like, for shipment to anywhere it is needed.

How to avoid the highly undesirable esterification reaction between acid and alcohol constituents in the blend is to use minimal heat effective to melt the fatty alcohol, keep water out of the blend and hot processing air to the extent feasible, and conduct the process rapidly, cooling the result of the melt-processing promptly. This not only confers a desirable spreading quality to the fatty alcohol, which has been discussed elsewhere—it also prevents esterification from occurring to any meaningful extent.

### CONCLUSION

It is tempting to say that the gist of the present invention reduces down to just substituting a dicarboxylic acid, preferably oxalic acid, in place of the sulfuric acid formerly disclosed for acidifying crushed gypsum combined with a fatty alcohol evaporation retardant. The invention as claimed, however, is not properly reduced down to its gist, apparently, and so must not be defined except by the claims to follow, with due allowance for consulting the whole specification and for giving consideration to what equivalents the inventor means also to be reasonably covered.

What I claim as new is:

1. For an unemulsified and substantially dry evaporation retardant composition in the form of a powder essentially comprising composite particles made by melt-processing together filmforming and non-filmforming chemicals, wherein said composition is intended to spread very rapidly in contact with water more effectively than if it did not contain any non-filmforming chemicals, and wherein said composition is intended to effectively distribute non-filmforming chemicals that are of benefit to plant culture, animal husbandry, or both, the characterizing inclusion in said composition of:
chemical means for conserving water by formation of an evaporation retardant film, exclusive of any variety of said film that significantly impairs water infiltration into soil if applied as a powder or an emulsion onto said soil;
bulking agent means for assisting to build composite particles formed out of a melt containing filmforming and non-filmforming chemicals;
chemical means for water-induced ionization of composite particles whereby they repel one another, and;
chemical means for benefitting plant culture, animal husbandry, or both, exclusive of any said benefitting means that merely conserves water, conditions soil by adding gypsum thereto, or both, doing substantially nothing else.

2. A product in accordance with claim 1, wherein said chemical means for water-induced ionization is a dicarboxylic acid.

3. A product in accordance with claim 2, wherein said dicarboxylic acid is oxalic acid.

4. A product in accordance with claim 1, wherein said chemical means for conserving water is a straight-chain, unbranched, fatty alcohol that is normally solid phase at room temperature, and said chemical means for water-induced ionization is a dicarboxylic acid.

5. A product in accordance with claim 4, wherein said dicarboxylic acid is oxalic acid.

6. A product in accordance with any preceding claim, in the alternative, wherein said bulking agent means for building composite particles, and said chemical means for benefitting plant culture, animal husbandry, or both, are provided by one and the same substance.

7. A product in accordance with claim 1, wherein said bulking agent means is microspheres.

8. A product in accordance with claim 1, wherein a long chain fatty acid such as tetradecanoic acid is added up to 5 percent of the film former.

9. A product in accordance with claim 1. where a low density absorption bead containing a useful material such as fertilizer, insecticide, bateriocide, growth hormone and the like may be admixed after the melt-mixed powder is produced so as to be pushed along with the spreading powder.
